(19) United States
(12) Patent Application Publication
(10) Pub. No.: US 2024/0120043 A1
(43) Pub. Date: Apr. 11, 2024

(54) OPTIMIZED CLASSIFICATION MODELS BASED ON LARGE PATIENT DATASETS TO IMPROVE MEDICAL CARE

(71) Applicant: Cornell University, Ithaca, NY (US)

(72) Inventors: Rainu Kaushal, New York, NY (US); Yongkang Zhang, New York, NY (US)

(73) Assignee: Cornell University, Ithaca, NY (US)

(21) Appl. No.: 18/538,169

(22) Filed: Dec. 13, 2023

Related U.S. Application Data
(63) Continuation of application No. 17/229,410, filed on Apr. 13, 2021.
(60) Provisional application No. 63/008,982, filed on Apr. 13, 2020.

(51) Int. Cl. G16H 10/60; G16H 10/40; G16H 50/30

(52) U.S. Cl. CPC G16H 10/60; G16H 10/40; G16H 50/30

(57) ABSTRACT

A computer implemented method can optimize classification of patients from large patient datasets. The method includes extracting, from data structures of different data sources, data related to a large plurality of patient. Patient electronic health record (EHR) data are linked across the data sources in a privacy preserving manner to generate patient information for the patient. Based on patient information for a selected patient, a high-cost status, a phenotype, and a persistence property of the selected patient are generated. The persistence property is one or both of a persistently high cost or a persistently high utilization. Furthermore, a high-cost status, a phenotype, and a persistence property are applied to a machine learning model to determine at least one risk score for the selected patient. The machine learning model is trained using training data related to the large plurality of patients.

### CROSS REFERENCE TO RELATED APPLICATIONS

This application is a continuation of U.S. Nonprovisional Patent Application No. 17/229,410, filed Apr. 13, 2021, which claims the priority benefit of U.S. Provisional Patent Application No. 63/008,982 filed Apr. 13, 2020, the contents of which are hereby incorporated by reference in their entirety as though fully set forth herein.

### TECHNICAL FIELD

The subject matter described herein relates to a devices, methods, and systems for identifying and classifying patient populations into actionable phenotypes. This patient classification system has particular but not exclusive utility for improving the quality and reducing the costs of medical care.

### BACKGROUND

Medical patients may currently be classified by systems, taxonomies, and nomenclature. For example, current clinical and functional groups may be derived exclusively from medical claims data (e.g., Medicare, Medicaid, and private insurance claims), and may include (1) children with complex needs, (2) non-elderly disabled, (3) patients with multiple chronic conditions, (4) patients with major, complex chronic conditions, (5) frail elderly, (6) patients with advancing illness, (7) patients with behavioral health factors, and (8) patients with social risk factors. However, claims data can have high latency (e.g., more than one year) and be time-consuming to access, and can lack longitudinal insight that is critical in predicting patient outcomes and determining care interventions. Furthermore, these patient groupings are not actionable, in that there are no specific clinical interventions associated with each grouping. Such groupings are also mutually exclusive, such that a patient cannot belong to more than one group. This approach may not fully capture the complexity of patients' medical status or the totality of their needs, as patients (especially high-cost patients) may have complex combinations of medical, behavioral, and social conditions.

Furthermore, current systems do not include predictive models or mechanisms for identifying patients who are not presently high cost, but who will be in the future.

Improving care for high-cost patients requires a better understanding of their characteristics and an actionable taxonomy to target effective interventions. Thus, it is to be appreciated that such commonly used classification systems have numerous drawbacks, including long latency, poor longitudinal insight, lack of actionable insight, mutually exclusive segments, and otherwise. Accordingly, long-felt needs exist for new devices, methods, and systems that address the forgoing and other concerns.

The information included in this Background section of the specification, including any references cited herein and any description or discussion thereof, is included for technical reference purposes only and is not to be regarded as subject matter by which the scope of the disclosure is to be bound.

### SUMMARY

Disclosed is a system for identifying and classifying high-cost patients and patient populations into actionable. computable phenotypes. The system includes a computer implemented method for identifying and categorizing high-cost and high-need high-cost (HNHC) patients into clinically meaningful, actionable patient categories. These categories can then be used to determine appropriate interventions. The categories are based on data extracted from electronic health records (EHR) from a single health system, insurance claims (e.g., Medicare, Medicaid, or private insurance claims), EHR data from multiple health systems through National Patient-Centered Clinical Research Network (PCORnet), and census data. Other online sources may be used as well, particularly for a patient's exposome including but not limited to the INSIGHT Clinical Research Network. The extracted data includes but is not limited to death data, diagnoses, medication orders, demographics, claims, patient-reported outcomes, geocodes, laboratory test results, or procedures.

Based on individual patient characteristics (as defined by the data), patients are statistically determined to be high-cost or non-high-cost, and are statistically mapped to one or more of 10 different actionable patient categories or phenotypes, and in some cases may be further categorized as “persistently high cost” and/or “persistently high preventable utilization.”. Based on these identified categories or phenotypes, patients may be recommended for at least one of five different intervention categories.

The present system can generate a taxonomy with clinically meaningful patient categories for high-cost or HNHCMedicare patients, for example, identifying those in the top 10% of total health spending. The system can compare patient characteristics and determine the likelihood of being a high-cost or HNHC patient across categories. For one example patient population (subsequently confirmed by a second patient population), the system identified ten non-mutually exclusive patient categories, including: multiple chronic conditions, single high cost chronic conditions, end-stage renal disease (ESRD), serious mental illness, opioid use disorder (OUD), seriously ill, single condition with high pharmacy cost, socially vulnerable, frail, and chronic pain. The majority of high-cost or HNHC patients had multiple chronic conditions (97.4%), followed by seriously ill (53.7%), and frail (48.9%). Patients falling into multiple categories were more likely to be high-cost or HNHC patients than those in a single category. The high-cost or HNHC patients can be highly heterogeneous with various medical and social conditions. Mapping high-cost or HNHC patients into clinically meaningful and actionable categories incorporating rich behavioral, social, and clinical factors could help health systems to identify and target appropriate interventions fitting the needs of high-cost or HNHC patients, including medical care services, behavioral health services, palliative care, pharmaceutical pricing policies, social services, or a combination of these services. To ensure that our findings can be applied to overall patients in the nation, we conducted a query using national data across all Clinical Research Networks (CRNs) affiliated with PCORnet. We found that the results are consistent across all CRNs, indicating that our findings can be applied to more and broader patient populations than those already examined.

Supplying this information to care providers and/or care coordinators may reduce unnecessary or preventable utilization of care services, and thus reduce costs. Furthermore, unlike current systems, the patient classification system disclosed herein can include predictive models or mechanisms for identifying patients who are not presently high cost, but who will be in the future. Such patients may be particularly likely to experience future high cost, HNHC, and safety problems, based on present-day classification, whereas analysis according to the present disclosure may help health systems develop preventive interventions to reduce unnecessary utilization and improve quality

The patient classification system disclosed herein has particular, but not exclusive, utility for improving the quality and reducing the costs of medical care. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect of the patient classification system includes a computer implemented method for classifying a medical patient. The computer implemented method includes extracting data related to the patient from one or more data structures and analyzing the data. The computer implemented method also includes based on the analyzing, determining a high-cost status of the patient. The computer implemented method also includes based on the analyzing, mapping the data to a phenotype of the patient. The computer implemented method also includes mapping the patient phenotype to at least one action category for the patient. The computer implemented method also includes based on the analyzing, computing a persistence property of the patient. The computer implemented method also includes based on the analyzing, the phenotype, the high-cost status, and the persistence property of the patient, computing at least one risk score of the patient. Other embodiments of this aspect include corresponding computer systems, apparatus, and computer programs recorded on one or more computer storage devices, each configured to perform the actions of the methods.

Implementations may include one or more of the following features. The computer implemented method further including writing the phenotype, at least one action category, high-cost status, persistence property, or at least one risk score into an electronic health record of the patient. In some embodiments, the one or more data structures include at least one of death data, diagnoses, medication orders, demographics, claims, patient-reported outcomes, geocodes, lab results, or procedures. In some embodiments, the one or more data structures further include at least one of a social determinant, a tumor registry, a biosample, a genomic result, a processed natural language input, or patient-generated data. In some embodiments, the data structures are accessed through at least one of electronic health records, insurance claims, national patient-centered clinical research network (PCORnet), or census data. In some embodiments, the patient phenotype is socially vulnerable, frail, end stage renal disease, single high-cost chronic condition, multiple chronic conditions, chronic pain, serious mental illness, opioid use disorder, seriously ill, or single condition with high pharmacy cost. In some embodiments, the at least one action category includes at least one of social services, medical care services, behavioral health services, palliative care, or pharmacological pricing policies. In some embodiments, the patient phenotype is socially vulnerable, and the at least one action category includes social services; or the patient phenotype if frail, and the at least one action category includes social services and medical care services; or the patient phenotype is end stage renal disease, and the at least one action category includes medical care services; or the patient phenotype is single high-cost chronic condition, and the at least one action category includes medical care services; or the patient phenotype is multiple chronic conditions, and the at least one action category includes medical care services; or the patient phenotype is chronic pain, and the at least one action category includes medical care services and behavioral health services; or the patient phenotype is serious mental illness, and the at least one action category includes behavioral health services; or the patient phenotype is opioid use disorder, and the at least one action category includes behavioral health services; or the patient phenotype is seriously ill, and the at least one action category includes palliative care; or the patient phenotype is single condition with high pharmacy cost, and the at least one action category includes pharmaceutical pricing policies. The computer implemented method further including: based on the analyzing, mapping the data to a second phenotype of the patient; and mapping the second phenotype of the patient to a second one or more action categories; and based on the analyzing, the phenotype, the second phenotype, the high-cost status, and the persistence property of the patient, computing the at least one risk score of the patient. In some embodiments, the high cost status of the patient includes high cost, future high cost, or non high cost, and the persistence property of the patient includes persistently high cost, persistently high preventable utilization, persistently high cost and persistently high preventable utilization, or non-persistent. Implementations of the described techniques may include hardware, a method or process, or computer software on a computer-accessible medium.

One general aspect includes a system including a processor configured to extract data related to a patient from one or more data structures; analyze the data; based on the analyzing, determine a high-cost status of the patient; based on the analyzing, map the data to a phenotype of the patient; map the patient phenotype to at least one action category for the patient; based on the analyzing, compute a persistence property of the patient; based on the analyzing, the phenotype, the high-cost status, and the persistence property of the patient, compute at least one risk score of the patient. Other embodiments of this aspect include corresponding computer systems, apparatus, and computer programs recorded on one or more computer storage devices, each configured to perform the actions of the methods.

Implementations may include one or more of the following features. The system where the processor is further configured to write the phenotype, at least one action category, high-cost status, persistence property, or at least one risk score into an electronic health record of the patient. In some embodiments, the patient phenotype is socially vulnerable, frail, end stage renal disease, single high-cost chronic condition, multiple chronic conditions, chronic pain, serious mental illness, opioid use disorder, seriously ill, or single condition with high pharmacy cost. In some embodiments, the at least one action category includes at least one of social services, medical care services, behavioral health services, palliative care, or pharmacological pricing policies. In some embodiments, the patient phenotype is socially vulnerable, and the at least one action category includes social services; or the patient phenotype if frail, and the at least one action category includes social services and medical care services; or the patient phenotype is end stage renal disease, and the at least one action category includes medical care services; or the patient phenotype is single high-cost chronic condition, and the at least one action category includes medical care services; or the patient phenotype is multiple chronic conditions, and the at least one action category includes medical care services; or the patient phenotype is chronic pain, and the at least one action category includes medical care services and behavioral health services; or the patient phenotype is serious mental illness, and the at least one action category includes behavioral health services; or the patient phenotype is opioid use disorder, and the at least one action category includes behavioral health services; or the patient phenotype is seriously ill, and the at least one action category includes palliative care; or the patient phenotype is single condition with high pharmacy cost, and the at least one action category includes pharmaceutical pricing policies. In some embodiments, the processor is further configured to: based on the analyzing, map the data to a second phenotype of the patient; and map the second phenotype of the patient to a second one or more action categories; and based on the analyzing, the phenotype, the second phenotype, the high-cost status, and the persistence property of the patient, compute the at least one risk score of the patient. In some embodiments, the high cost status of the patient includes high cost, future high cost, or non high cost, and the persistence property of the patient includes persistently high cost, persistently high preventable utilization, persistently high cost and persistently high preventable utilization, or non-persistent. Implementations of the described techniques may include hardware, a method or process, or computer software on a computer-accessible medium. Other embodiments of this aspect include corresponding computer systems, apparatus, and computer programs recorded on one or more computer storage devices, each configured to perform the actions of the methods.

Implementations may include one or more of the following features. The system where the one or more data structures further include at least one of a social determinant, a tumor registry, a biosample, a genomic result, a processed natural language input, or patient-generated data. In some embodiments, the data structures are accessed through at least one of electronic health records, insurance claims, PCORnet, or census data. Implementations of the described techniques may include hardware, a method or process, or computer software on a computer-accessible medium.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the patient classification system, as defined in the claims, is provided in the following written description of various embodiments of the disclosure and illustrated in the accompanying drawings.

### BRIEF DESCRIPTION OF THE DRAWINGS

Illustrative embodiments of the present disclosure will be described with reference to the accompanying drawings, of which:

FIG. 1 is a chart illustrating an exemplary sample selection process for establishing patient categories and their relative prevalence or probability within a population, in accordance with the present embodiments.

FIG. 2A is an exemplary representation of the patient characteristics of high-cost patients vs. non-high-cost patients, by patient categories, in accordance with the present embodiments.

FIG. 2B is an exemplary representation of the patient characteristics of high-cost patients vs. non-high-cost patients, by patient categories, in accordance with the present embodiments.

FIG. 2C is an exemplary representation of the patient characteristics of high-cost patients vs. non-high-cost patients, by patient categories, in accordance with the present embodiments.

FIG. 3 shows an exemplary mapping of high-cost patients into categories or phenotypes, in accordance with the present embodiments.

FIG. 4 shows the likelihood of a patient from the selected population being an HNHC patient in each patient category or phenotype, in accordance with the present embodiments.

FIG. 5 shows the number of categories or phenotypes into which each high-cost patient is classified, in accordance with the present embodiments.

FIG. 6A shows an exemplary distribution of high-cost patients and the likelihood of being a high-cost patient across categories are similar with our primary analysis after excluding Part D costs, in accordance with the present embodiments.

FIG. 6B shows an exemplary distribution of high-cost patients and the likelihood of being a high-cost patient across categories are similar with our primary analysis after excluding Part D costs, in accordance with the present embodiments.

FIG. 7A shows an exemplary distribution of high-cost dual-eligible patients into categories or phenotypes, in accordance with the present embodiments.

FIG. 7B shows exemplary characteristics of the patient population of FIG. 7B, in accordance with the present embodiments.

FIG. 8A shows the likelihood of being a high-cost patient in each patient category of an example patient population, in accordance with the present embodiments.

FIG. 8B shows the number of categories each high-cost patient falls into, among the example population of FIG. 8A, in accordance with the present embodiments.

FIG. 9 shows an exemplary mapping of patient categories or phenotypes to action categories, in accordance with the present embodiments.

FIG. 10 shows a flow diagram of an example computer-implemented patient classification method, in accordance with the present embodiments.

FIG. 11 is a schematic representation, in block diagram form, of an example network architecture over which the method of FIG. 10 may operate, in accordance with the present embodiments.

FIG. 12 is a schematic diagram of a processor circuit, according to the present embodiments.

FIG. 13 is a table showing example data types and the example data sources from which they may be available, in accordance with the present embodiments.

### DETAILED DESCRIPTION

In accordance with the present embodiments, a patient classification system is provided for identifying and categorizing high-cost patients into clinically meaningful, actionable patient categories. These categories can then be used to determine appropriate interventions for cost reduction and quality improvement. The categories or phenotypes are based on data extracted from individual and networks (i.e., PCORnet) of electronic health records (EHR), insurance claims, and census data, although in some cases the categories may be identified or implemented using EHR alone. The extracted data can include, but is not limited to, death data, diagnoses, medication orders, demographics, physical addresses, vital signs, claims (Medicare, Medicaid, private insurance, etc.), patient-reported outcomes, geocodes, laboratory testing results, or procedures. In some embodiments, data may also come from social determinants of health, exposome, tumor registry, biosamples, genomic results, natural language processing, or patient-generated data.

Based on individual patient characteristics (as defined by the extracted data), patients are statistically determined to be probable high-costor HNHC patients or probable non-high-cost or non-HNHC patients. Probable high-cost or HNHC patients are then statistically mapped to one or more actionable categories or phenotypes. In an example, the patient may be categorized with one or more of ten different phenotypes: “socially vulnerable”, “frail”, “end stage renal disease”, “single high-cost chronic condition”, “chronic pain”, “serious mental illness”, “opioid use disorder”, “seriously ill”, or “single condition with high pharmacy cost”, or combinations thereof. For example, probable high-cost or HNHC heart failure patients may map to the “frail” category, whereas probable high-cost or HNHC congestive heart failure patients may map to the “seriously ill” category, each of which prescribes different interventions. Social vulnerability may be determined by neighborhood-level social determinants of health data, such as median income, unemployment rate, income disparity, poverty rate, education, public assistance, crowding housing conditions, cost of living, or other data, or composite social indices derived therefrom. These data can be extracted at the zip code, census block groups, or other geographic level from the American Community Survey data or other sources. Example social indices known in the art include but are not limited to Area Deprivation Index (ADI), Social Deprivation Index (SDI), Social Vulnerability Index (SVI), or Neighborhood Stress Score (NSS). In some embodiments, the area deprivation index (ADI) may be preferred, as it can be indexed using information available in a patient's EHR, and (for example) patients within the top 30% of ADI scores may be identified as socially vulnerable patients.

In various embodiments, high-cost or HNHC patients may be further categorized as “persistently high cost”, “persistently high incidence of preventable resource utilization.”, or “double persistent” (e.g., persistently high cost and preventable utilization). It is noted that “double persistent” patients are only 1.2% of the Medicare population, but represent 26% of all preventable utilization, and therefore may offer disproportionate opportunities for cost reduction based on improvements in care. Preventable utilization may for example include preventable emergency department (ED) visits, preventable ambulatory care sensitive conditions admissions, and unplanned 30-day readmissions.

Where costs are not directly available from the data, costs may be determined analytically by converting utilization (e.g., procedures, prescriptions, office visits) to cost based on standard or probable costs.

Each patient category or phenotype is then mapped to an action category or intervention. In an example, there are five different action categories or interventions: “social services”, “medical care services”, “behavioral health services”, “palliative care”, and “pharmacological pricing policies”, or combinations thereof. Each intervention aims to address health issues that patients in a category may have to improve quality and reduce unnecessary utilization.

When the patient category or phenotype is visible to a care provider or care coordinator (e.g., as part of the patient's EHR data), along with the recommended action category, it becomes much easier for the care provider or care coordinator to understand the nature and severity of the patient's condition and potentially effective interventions, and thus they can align one or more intervention to each patient category to address the following problems:
(1) Reduce unnecessary/preventable utilization of care services.
(2) Reduce persistence of high cost patients across multiple years.
(3) Reduce persistence of preventable utilization across multiple years.
(4) Reduce “double persistence” of high cost and high preventable utilization.

The patient classification system disclosed herein addresses the clinical, behavioral, and social complexity of high-cost or HNHC patients with clinically meaningful categories or phenotypes that permit targeted interventions that incorporate the perspectives of multiple stakeholders, including the patient, while being data driven. The present disclosure aids substantially in the operation of electronic health record (EHR) systems to manage patient care, by improving the information content of the EHR without substantially increasing the time required to generate, store, retrieve, process, or display the EHR or requiring additional data elements from the EHR. Implemented on a processor or computer system in communication with data structures accessible via a network, the patient classification system disclosed herein provides practical improvement in medical care and the computers associated with electronic health records. This improved classification system transforms an EHR containing discrete medical information into one that also contains an actionable classification of the patient and their care needs, without the normally routine need to question the patient. In some cases, this may involve analyzing or processing large amounts of data from diverse sources in real time or near real time. This unconventional approach improves the functioning of the EHR system, by improving its information content without adding undue burden to care providers.

The patient classification system may be implemented as a decision tree with outputs viewable on a display, and operated by a control process executing on a processor that accepts user inputs from a keyboard, mouse, or touchscreen interface, and that is in communication with one or more databases. In that regard, the control process performs certain specific operations in response to different inputs or selections made at different times or in response to different inputs. Certain structures, functions, and operations of the processor, display, sensors, and user input systems are known in the art, while others are recited herein to enable novel features or aspects of the present disclosure with particularity.

These descriptions are provided for exemplary purposes only, and should not be considered to limit the scope of the patient classification system. Certain features may be added, removed, or modified without departing from the spirit of the claimed subject matter.

High-cost or HNHC patients are a small group of individuals with major health problems and account for a disproportionate share of health care utilization. These patients are more likely to interact with the health system, incur preventable health costs, and suffer quality and safety problems as well as poorer health outcomes. The concentration of spending among high-cost or HNHC patients has motivated payers and providers to design new care models to better meet their needs, improve quality, and reduce unnecessary utilization. However, the majority of these care models focus on medical services, such as through care managers.

High-cost or HNHC patients are not a homogenous group, but rather, have varied medical conditions, functional limitations, and social circumstances. A single set of services may not meet the needs of all high-cost or HNHC patients. Refined understanding of which patients may benefit from which types of interventions is needed. While evidence suggests programs can be tailored for groups of patients with shared characteristics, doing so may require rigorously developing categories of patients from varied data sources beyond administrative data and designing care models accordingly.

Taxonomies can provide insights for categorizing high-cost or HNHC patients, but can have practical challenges that may limit the extent to which health systems can match care delivery models with particular groups of patients. First, mutually exclusive segments may not effectively capture the totality of a patient's needs. For example, patients with serious mental illness likely incur higher costs than those without in a given segment. Second, most studies have relied heavily on administrative data — usually Medicare claims data—but administrative data alone may fail to capture important aspects of patients' clinical circumstances, such as functional limitations, illness severity, and response to therapy. Third, these taxonomies do not robustly incorporate socioeconomic characteristics, which have a strong relationship with healthcare utilization. Furthermore, some studies purely used data-driven methods (e.g., cluster analysis) to develop patient categories. It is not clear if these categories are clinically meaningful from care managers or clinicians' perspectives.

The present system can include a new taxonomy with ten non-mutually exclusive patient categories to understand the medical and social complexity of high-cost patients. These categories can be conceptualized through literature review, data-driven insights, and stakeholder input including patients. The system can operationalize these categories using a dataset that included claims, clinical data, and social risk factors.

In an example, a retrospective cohort study is performed to identify and categorize high-cost Medicare beneficiaries into ten non-mutually exclusive patient categories using Medicare claims, clinical data from the New York City INSIGHT network (part of PCORnet), and social determinants of health data from the American Community Survey (ACS). The system examined the percentage of high-cost or HNHC patients captured by each of these categories and the characteristics of patients within them. The study then analyzes the likelihood that patients in a given category will be high cost or HNHC.

The example primary analysis included 428,024 Medicare fee-for-service beneficiaries continuously enrolled in Medicare Part A and Part B in 2013. Beneficiaries were excluded if they were 1) dually-eligible because their cost information was not completely captured by Medicare claims (we performed a sensitivity analysis for the dual-eligible patients), 2) had any managed care participation, or 3) died during the year as their limited months of enrollment may result in artificially low costs.

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

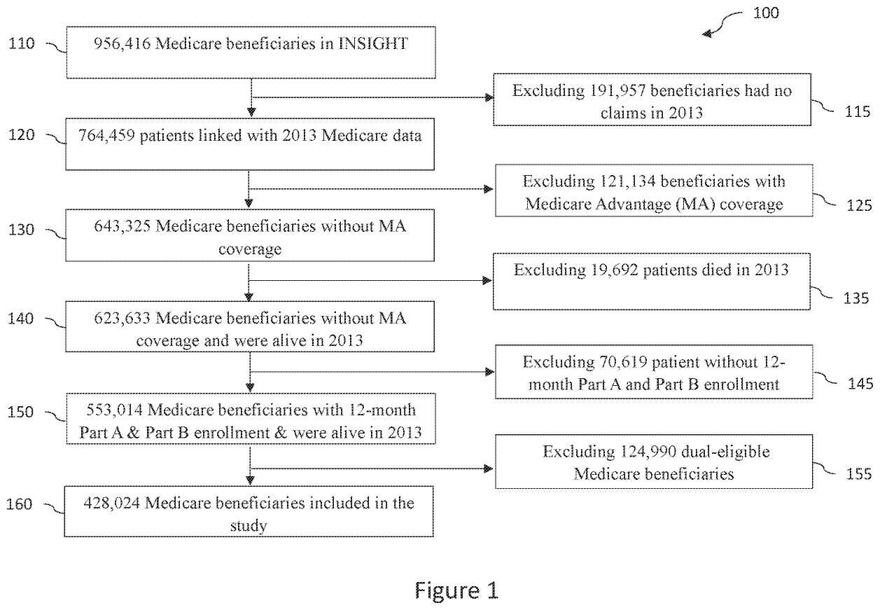

FIG. 1 is a chart illustrating an exemplary sample selection process for establishing patient categories and their relative prevalence or probability within a population, in accordance with the present embodiments. Data sources may include clinical data from electronic health records (EHRs), Medicare fee-for-service claims, and community-level social determinants of health data. In this example, clinical data were obtained from INSIGHT. The Patient Centered Outcome Research Institute (PCORI) funded INSIGHT aggregates clinical data from seven independent health systems in New York City, including the Clinical Directors Network, Mount Sinai Health System, Montefiore Medical Center & Albert Einstein Medical College, NYU Langone Medical Center, Columbia University Vagelos College of Physicians and Surgeons, New York Presbyterian Hospital/Columbia (NYP West), New York-Presbyterian Hospital/Cornell (NYP East), and Weill Cornell Medicine (the multispecialty faculty practice of Weill Cornell Medical College). Medicare claims included those for Parts A and B, in addition to drug claims for Part D. We merged the clinical data from the NYC-CDRN with Medicare claims using a crosswalk developed by NYC-CDRN. Finally, neighborhood social determinants of health data at the US census block group level from ACS were merged with Medicare claims and EHR data.

The development of the high-cost or HNHC patient categories was based on a combination of qualitative and quantitative results. A high-cost or HNHC patient category was included if it fit the following criteria: (1) it had good face validity: it was prioritized by literature and/or by physicians, health system executives, and patients during structured interviews and focus groups; (2) it was measurable: a category could be measurable using administrative, clinical, or social determinants of health data; (3) it had good internal validity: a category could represent a group of patients with shared characteristics and needs and the average healthcare spending was higher than patients not fitting into any high-cost or HNHC patient categories.

To develop a taxonomy for high-cost or HNHC patients with good face validity, the survey started with a literature review to identify high-cost or HNHC patient categories that have been identified in the previous research. To test the internal validity, the system conducted a data driven preliminary analysis to test the validity of the high-cost or HNHC patient groups identified from the literature and focus groups and interviews. Using a Medicare dataset including 1.8 million Medicare beneficiaries in New York State and 2.2 million Medicare beneficiaries in Texas of 2012, we first examined (1) if a high-cost or HNHC category could be electronically measured using our rich, diverse data sources and (2) if patients included in a high-cost or HNHC category had shared characteristics and health needs.

The survey calculated the total spending of each beneficiary and considered an individual high-cost if he or she fell into the top 10% of total spending. In some embodiments, a patient may be identified as a high-need of he or she falls into the top 10% of total utilization. In developing the categories, the system examined (1) the completeness of capture and distribution of high-cost or HNHC patients across these categories; (2) the distinctness across high-cost or HNHC categories; (3) the amount of healthcare spending across categories; and (4) spending for patients in high-cost or HNHC categories compared to all other patients.

The final taxonomy included ten non-mutually exclusive categories of high-cost or HNHC patients, including (1) Frail; (2) end-stage renal disease (ESRD); (3) single high cost chronic condition; (4) multiple chronic conditions; (5) chronic pain; (6) serious mental illness; (7) opioid use disorder; (8) seriously ill; (9) single high cost chronic conditions; and (10) socially vulnerable.

The first nine clinical categories were based on diagnoses, procedures, and health care utilization. To measure the socially vulnerable category, we created a census block group level Social Vulnerability Index (SVI) using data from ACS and a previously developed algorithm. The system defined socially vulnerable patients as those living in a census block group that is in the top 30% in terms of the SVI score. Detailed descriptions of these patient categories are available in the below tables.

TABLE 1
Definition and Computable Phenotypes

| | Measures and Data Sources | | |
|---|---|---|---|
| | | | Social |
| Computable | | | determinants |
| phenotypes | Claims data | Clinical data | of health data |
| Seriously ill | >=1 seriously | Seriously ill | — |
| | ill indicator | with low | |
| | | albumin | |
| | | Seriously ill | |
| | | with low | |
| | | BMI | |
| Multiple chronic | >= 3 out of | — | — |
| conditions | the 25 CCW | | |
| | chronic | | |
| | conditions | | |
| Single chronic | HIV | HIV with AIDs | — |
| conditions (>=1 | | (CD 4 cell | |
| out of the three) | | count) | |
| | HCV (HCV | — | |
| | with cirrhosis) | | |
| | Sickle cell | — | |
| | Rheumatoid | — | — |
| | arthritis | | |
| Single | Multiple | | |
| conditions with | sclerosis | | |
| high pharmacy | Crohn's | | |
| cost (>=1 out | disease | | |
| of the three) | | | |
| <65 with disability | <65 with | — | — |
| or end-stage renal | disability or | | |
| disease (ESRD) | ESRD | | |
| >=65 with ESRD | >=65 with | — | — |
| | ESRD | | |
| Chronic pain | >=1 chronic | — | — |
| | pain condition | | |
| Frail | >=2 frail | Frail with | — |
| | indicators | low albumin | |
| | | Frail with | |
| | | low BMI | |
| | | Frail with | |
| | | extreme | |
| | | obesity | |
| Mental illness | >=1 serious | — | — |
| | mental health | | |
| | condition | | |
| Socially | | | Top 30% of |
| vulnerable | | | social |
| | | | vulnerability |
| | | | score |

TABLE 2
Conditions, procedures, lab tests, and other characteristics
used to define computable phenotypes

| Conditions, procedures, lab tests, and other | Computable |
|---|---|
| characteristics | Phenotype |
| Chronic Obstructive Pulmonary Disease (COPD) * | Seriously ill |
| Idiopathic fibrosing alveolitis/fibrosing alveolitis | |
| (IPFFA) * | |
| Non-small cell lung cancer stage IIIB or IV * | |
| Other primary malignancy that is metastatic to the | |
| lung | |
| Malignant pleural effusion | |
| Mesothelioma * | |
| Other interstitial lung disease w/non-steroid | |
| response * | |
| Sarcoidosis* | |
| Other malignancy * | |
| Chronic kidney disease (stage IV or V) * | |
| Congestive heart failure (CHF) * | |
| Amyotrophic lateral sclerosis (AES) * | |
| Any hospice | |
| Addition criteria for conditions with * | |
| Supplementation oxygen at home | |
| 2+ hospitalization in a year | |
| Severe protein malnutrition | |
| Frailty | |
| Hemodialysis (additional criterion for | |
| Chronic kidney disease) | |
| Ischemic heart disease (including acute | Multiple chronic |
| myocardial infarction) | conditions |
| Chronic kidney disease | |
| Heart failure | |
| Diabetes | |
| Stroke/transient ischemic attack | |
| Asthma | |
| Chronic obstructive pulmonary disease | |
| Depression | |
| Alzheimer's Disease, Related Disorders, | |
| or Senile Dementia | |
| Rheumatoid arthritis/osteoarthritis | |
| Cancer, breast | |
| Cancer, colorectal | |
| Cancer, endometrial | |
| Cancer, lung | |
| Cancer, prostate | |
| Cataract | |
| Glaucoma | |
| Benign prostatic hyperplasia | |
| Hypertension | |
| Anemia | |
| Hyperlipidemia | |
| Osteoporosis | |
| Acquired hypothyroidism | |
| Hip/pelvic fracture | |
| Atrial fibrillation | |
| Human immunodeficiency virus (HIV) | Single chronic |
| Hepatitis C (HCV) | condition |
| HCV plus Cirrhosis | |
| Sickle cell | |
| Rheumatoid arthritis | Single condition |
| Multiple sclerosis | with high |
| Crohn's disease | pharmacy cost |
| Beneficiaries' age under 65 | <65 with disability |
| | or ESRD |
| End-stage renal disease (ESRD) | >=65 with ESRD |
| Beneficiaries' age equal or over 65 | |
| Chronic pain due to trauma | Chronic pain |
| Chronic post-thoracotomy pain | |
| Other chronic postoperative pain | |
| Other chronic pain | |
| Chronic pain syndrome | |
| Abnormality of gait | |
| Abnormal loss of weight and underweight | Frail |
| Adult failure to thrive | |
| Cachexia | |
| Debility | |
| Difficulty in walking | |
| Fall | |
| Muscular wasting and disuse atrophy | |
| Muscle weakness | |
| Pressure ulcer | |
| Senility without mention of psychosis | |
| Durable medical equipment | |
| Depression | Mental illness |
| Bipolar Disorder | |
| Post-Traumatic Stress Disorder (PTSD) | |
| Schizophrenia and Other Psychotic Disorders | |
| Under weight, BMI < 18.5 | Seriously ill, frail |
| Extreme obesity, BMI >= 40 | Frail |
| Low albumin, albumin level < 2.0 | Seriously ill, frail |
| CD4 cell counts <200 to identify AIDS | Single chronic |
| | condition |
| Dialysis days in 2013 | <65 with |
| | disability or |
| | ESRD, >=65 |
| | with ESRD |
| % of people with high school or GED degree | Socially |
| GINI index | vulnerable |
| Respiratory hazard index | |

The taxonomy calculated standardized total Medicare spending for each beneficiary in 2013. High-cost or HNHC patients were defined as those with the highest 10% of total spending. The system mapped all Medicare beneficiaries and high-cost or HNHC patients into the ten patient categories. The system first compared the demographic characteristics and comorbidities between high-cost and non-high-cost or HNHC patients. The system calculated the percent of high-cost or HNHC patients captured by each patient category, as well as the likelihood that a patient in any given category would be high-cost or HNHC. The novel taxonomy allows a patient to fall into multiple categories if their conditions are highly complex. The system identified high-cost or HNHC patients in multiple categories and calculated the proportion of high-cost or HNHC patients in each pair of categories. The system presented the dominant category pairs that concentrate high-cost or HNHC patients.

To examine the healthcare utilization associated with vulnerable social conditions, the system identified 71,862 patients with their 9-digit zip codes available in New York State or New Jersey for a subgroup analysis. The system first mapped these patients to census block groups using a zip code/census block group crosswalk from a commercial source.

For some patient categories with relevant clinical markers, the system conducted subgroup analysis to identify patients at higher risk of being a high-cost or HNHC patient by incorporating laboratory tests and vital signs from clinical data and additional information from claims data. Based on clinicians' experience and literature review, the system identified patients who were underweight or with low albumin level (under 2 g/dl) in the serious illness category, HIV patients with AIDS, HCV patients with cirrhosis, or ESRD patients with any dialysis days. The system also identified patients with low albumin level, who were underweight (BMI<18.5), or who were extremely obese (BMI>=40) in the frail category.

Since not all beneficiaries have Part D coverage, the system redefined the high-cost or HNHC patients by dropping Part D cost and repeated the primary analysis. The system also did a sensitivity analysis for dual-eligible patients. All analyses were performed using SAS 9.4 and STATA MP 14.0. The Institutional Review Board at Weill Cornell Medicine approved this study.

A total of 42,802 high-cost or HNHC patients were identified from an initial sample of 428,024 Medicare beneficiaries. Demographic characteristics differed significantly between high-cost and non-high-cost patents (Table 1). Compared to non-high-cost patients, high-cost patients were more likely to be older (75.5 vs. 74.7, p<0.001), male (48.8% vs. 43.2% p<0.001), African American (8.6% vs. 7.5%, p<0.001), and have more chronic conditions (8.3 vs. 5.1, p<0.001). high-cost or HNHC patients were also more likely to have originally qualified for Medicare because of disability or ESRD. Average Medicare spending per beneficiary among high-cost patients was more than 8 times higher than for non-high-cost or non-HNHC patients ($68,481 vs. $8,234, p<0.001).

Before continuing, it should be noted that the examples described above are provided for purposes of illustration, and are not intended to be limiting. Other devices data, analysis methods, or categorization methods may be utilized to carry out the operations described herein.

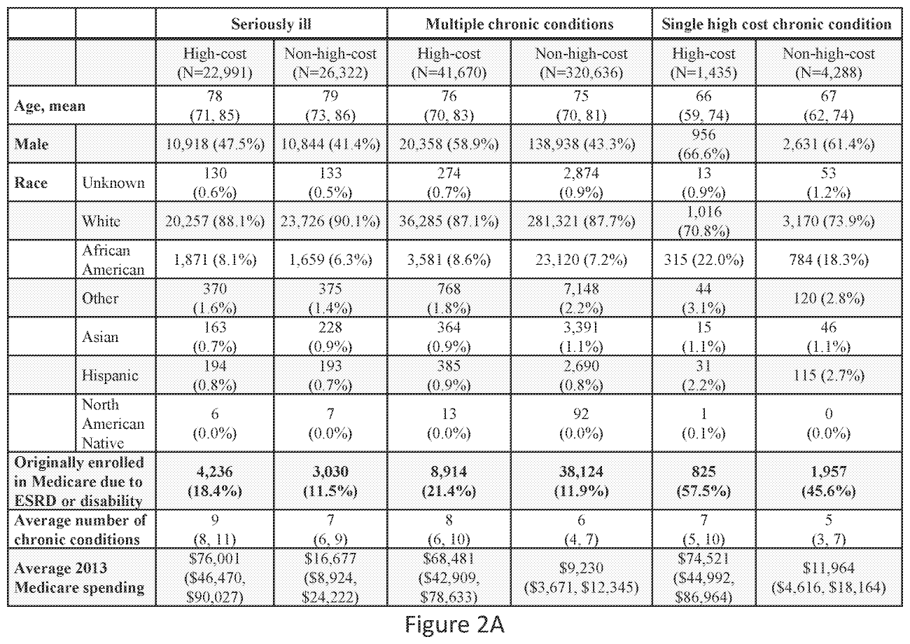

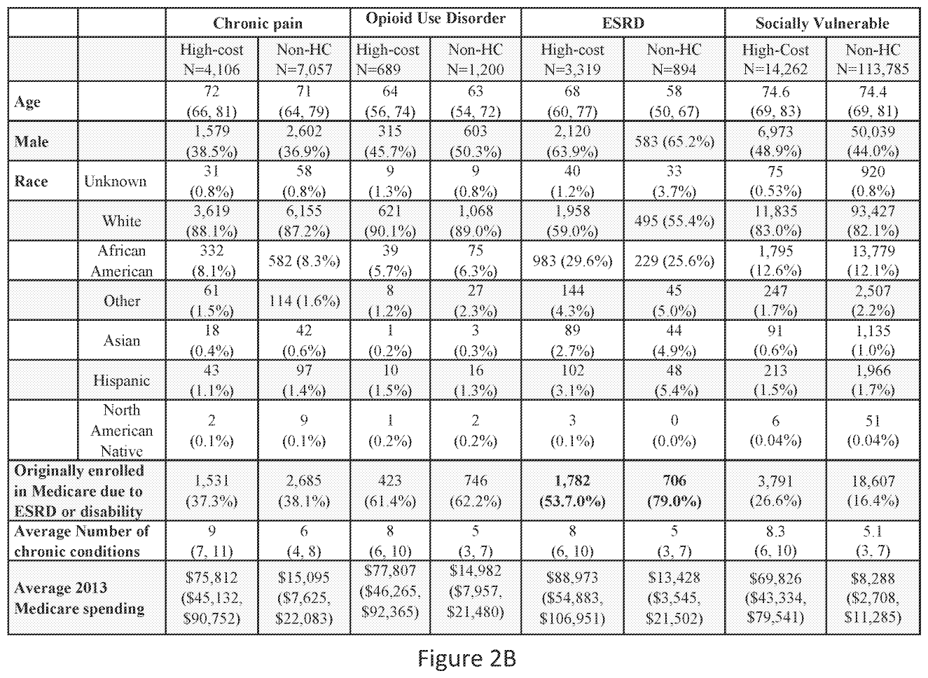

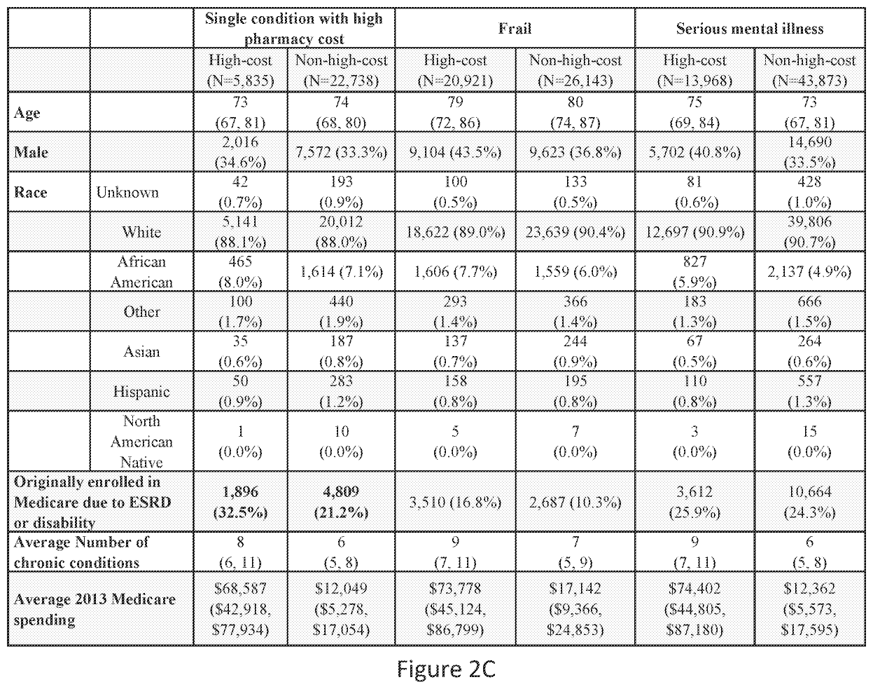

FIG. 2A is an exemplary representation of the patient characteristics of high-cost patients vs. non-high-cost patients, by patient categories, in accordance with the present embodiments.

FIG. 2B is an exemplary representation of the patient characteristics of high-cost patients vs. non-high-cost patients, by patient categories, in accordance with the present embodiments.

FIG. 2C is an exemplary representation of the patient characteristics of high-cost patients vs. non-high-cost patients, by patient categories, in accordance with the present embodiments. The characteristics of high-cost patients in each category also differed from non-high-cost patients. Among high-cost patients, 97.4% had multiple chronic conditions, 53.7% were seriously ill, 48.9% were frail, 32.6% had serious mental health issues, 13.6% had single condition with high pharmacy cost, 9.6% had chronic pain, 7.8% had ESRD, 3.4% had single high cost chronic condition, and 1.6% had opioid use disorder, as indicated in the below table. The ten clinical categories captured 99.0% of high-cost patients.

TABLE 3
Patient Categories by Percentage

| | Number of high-cost | % of high-cost |
|---|---|---|
| Patient | patients that fall | patients that fall |
| categories | into each category | into each category |
| Multiple chronic | 41,670 | 97.4% |
| conditions | | |
| Seriously ill | 22,991 | 53.7% |
| Frail | 20,921 | 48.9% |
| Serious mental | 13,968 | 32.6% |
| illness | | |
| Single condition | 5,834 | 13.6% |
| with high | | |
| pharmacy cost | | |
| Chronic pain | 4,106 | 9.6% |
| Patients with | 3,319 | 7.8% |
| ESRD | | |
| Single high cost | 1,435 | 3.4% |
| chronic condition | | |
| Opioid use | 689 | 1.6% |
| disorder | | |
| Patients not in | 441 | 1.0% |
| categories | | |
| Total | 42,802 | 100.0% |

The likelihood of being a high-cost patient varied considerably among categories For example, 78.8% of patients with ESRD were high-cost. By comparison, about half (44.5 to 46.6%) of patients who were seriously ill or frail were high-cost, and around 37% of patients in the chronic pain and the opioid use disorder category were high-cost. Patients in the remaining clinical categories had a relatively low probability of being high-cost.

As over 97% of high-cost patients had multiple chronic conditions, we excluded this category from the analysis of the overlap across categories and focused on high-cost patients falling into other categories.

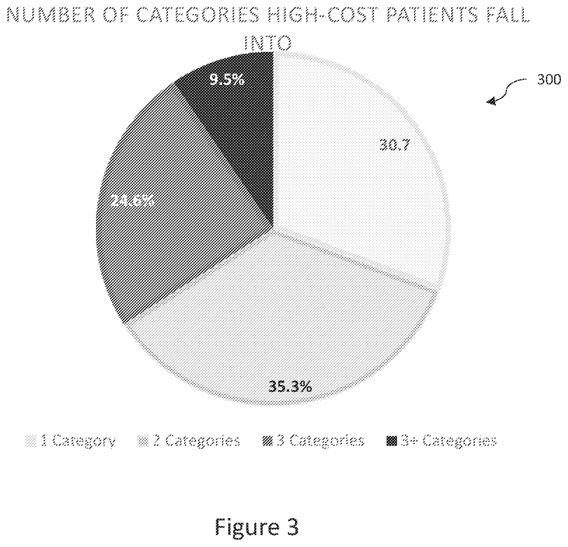

FIG. 3 is a chart 300 showing an exemplary mapping of high-cost patients into categories or phenotypes, in accordance with the present embodiments. Around 70% of high-cost patients were mapped into multiple categories, with 35.3% in two and 34.1% in three or more patient categories (FIG. 3). These patients were most highly concentrated in three pairs of categories: frail and seriously ill (49.7%), frail and serious mental illness (27.0%), and seriously ill and serious mental illness (26.3%).

We did not include multiple chronic conditions category as over 97% of high-cost patients were in this category. We only counted number of high-cost patients falling into each of the other eight clinical categories.

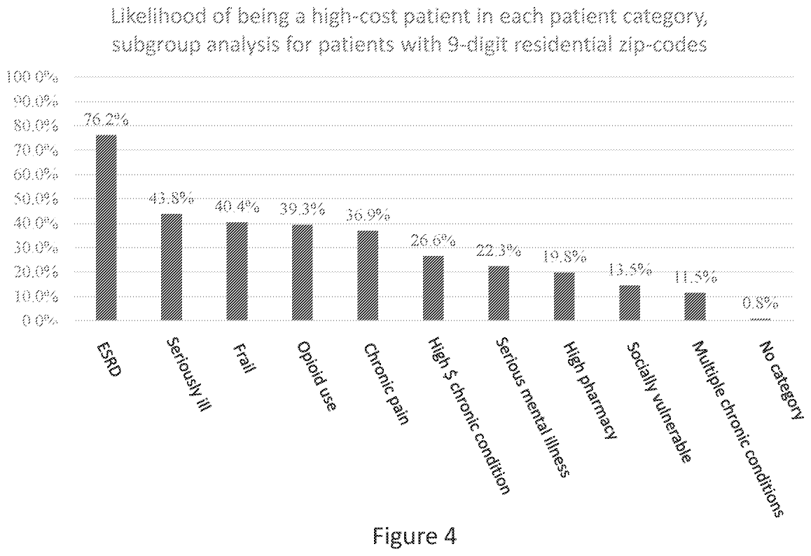

FIG. 4 shows the likelihood of a patient from the selected population being an high-cost patient in each patient category or phenotype, in accordance with the present embodiments.

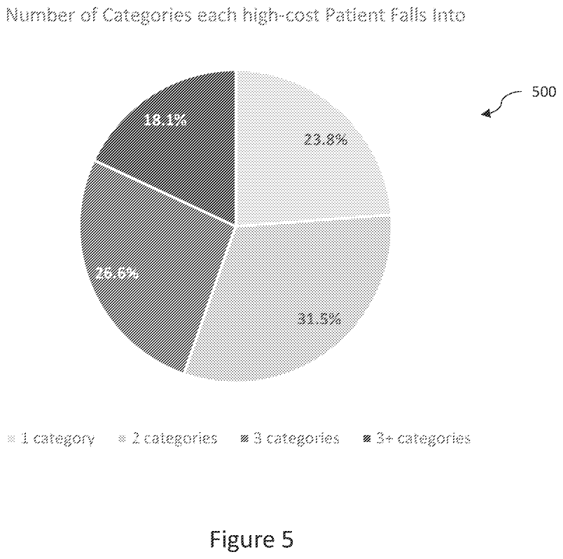

FIG. 5 is a chart 500 showing the number of categories or phenotypes into which each high-cost patient are classified, in accordance with the present embodiments.

We found similar results in our subgroup analysis for patients with 9-digit residential zip codes, as illustrated in the below tables. 13.5% of socially vulnerable patients were high-cost patients, representing 40.1% of overall high-cost patients in this sample. As we did for the overall patient population, we identified patients falling into multiple categories by additionally including the socially vulnerable category. We found 76.2% of high-cost patients were in multiple categories, with 31.5% in two and 44.6% in three or more patient categories (see for example FIGS. 4 and 5).

TABLE 4
Patient characteristics of high-cost vs. non-high-cost patients

| | | High-cost patients | | Non-high-cost patients | | |
|---|---|---|---|---|---|---|
| | | (N = 42,802) | | (N = 385,222) | | p value |
| Age, mean | | 75.5 | (69, 83) | 74.7 | (69, 81) | p < 0.001 |
| Male | | 20,878 | (48.8%) | 166,222 | (43.2%) | p < 0.001 |
| Race/Ethnicity | Unknown | 294 | (0.7%) | 3,966 | (1.0%) | p < 0.001 |
| | White | 37,216 | (87.0%) | 335,114 | (87.0%) | |
| | African American | 3,697 | (8.6%) | 28,716 | (7.5%) | |
| | Other | 802 | (1.9%) | 9,008 | (2.3%) | |
| | Asian | 377 | (0.9%) | 4,310 | (1.1%) | |
| | Hispanic | 403 | (0.9%) | 3,994 | (1.0%) | |
| | North AmericanNative | 13 | (0.0%) | 114 | (0.0%) | |
| Original reason | ESRD or disability | 9,461 | (22.1%) | 50,112 | (13.0%) | p < 0.001 |
| for Medicare | Other | 33,341 | (77.9%) | 335,110 | (86.7%) | |
| enrollment | | | | | | |
| Average number | | 8.3 | (6, 10) | 5.1 | (3, 7) | p < 0.001 |
| of chronic | | | | | | |
| conditions | | | | | | |

| | | | |
|---|---|---|---|
| Average 2013 | $68,481 | $ 8,234 | p < 0.001 |
| Medicare spending | ($42,880, $78,569) | ($2,789, $11,096) | |

Notes:
ESRD: end-stage renal disease; p values indicate the significance of the difference between the high cost group and non-high cost group. Parentheses for age, average number of chronic conditions, and average 2013 Medicare spending are interquartile intervals.

TABLE 5
Patient categories and number of high-
cost patients in each category

| | Number of high-cost | % of high-cost |
|---|---|---|
| Patient | patients that fall | patients that fall |
| categories | into each category | into each category |
| Multiple chronic | 6,947 | 96.7% |
| conditions | | |
| Seriously ill | 3,832 | 53.3% |
| Frail | 3,416 | 48.2% |
| Socially | 2,913 | 40.5% |
| Vulnerable | | |
| Serious mental | 2,474 | 34.4% |
| illness | | |
| Single condition | 1,085 | 15.1% |
| with high | | |
| pharmacy cost | | |
| Chronic pain | 708 | 9.9% |
| Patients with | 514 | 7.2% |
| ESRD | | |
| Single high cost | 343 | 4.8% |
| chronic condition | | |
| Opioid use | 129 | 1.8% |
| disorder | | |
| Patients not in | 58 | 0.8% |
| categories | | |
| Total | 7,186 | 100.0% |

Results for sensitivity analysis after excluding Part D costs and for dual-eligible patients were also calculated.

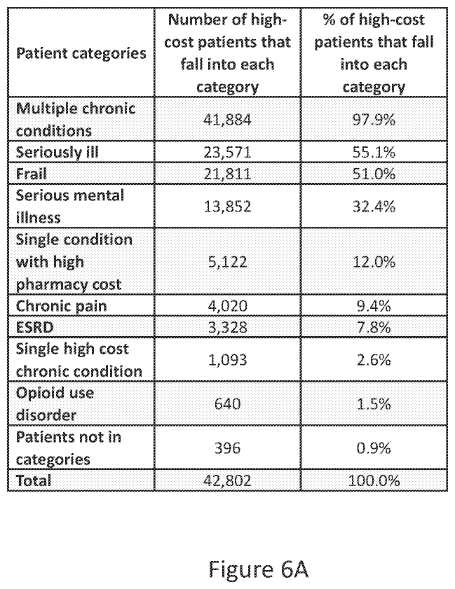

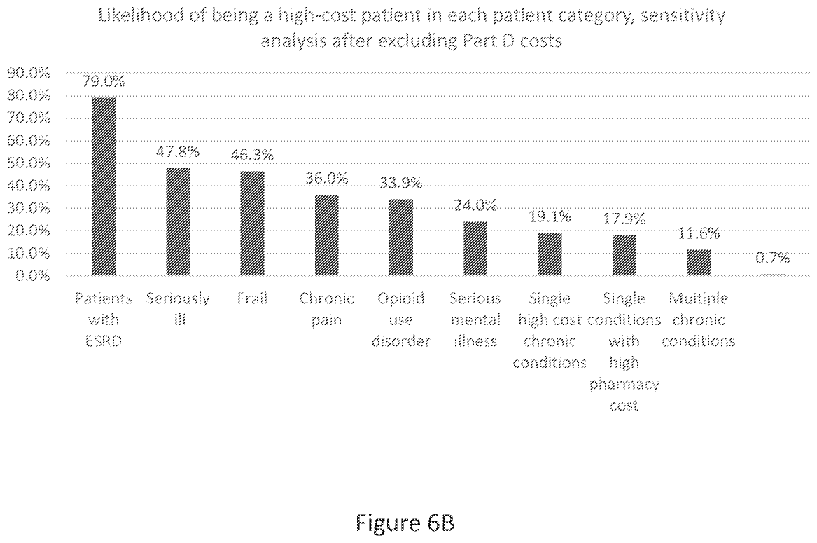

FIG. 6A shows an exemplary distribution of high-cost patients and the likelihood of being a high-cost patient across categories are similar with our primary analysis after excluding Part D costs, in accordance with the present embodiments.

FIG. 6B shows an exemplary distribution of high-cost patients and the likelihood of being a high-cost patient across categories are similar with our primary analysis after excluding Part D costs, in accordance with the present embodiments.

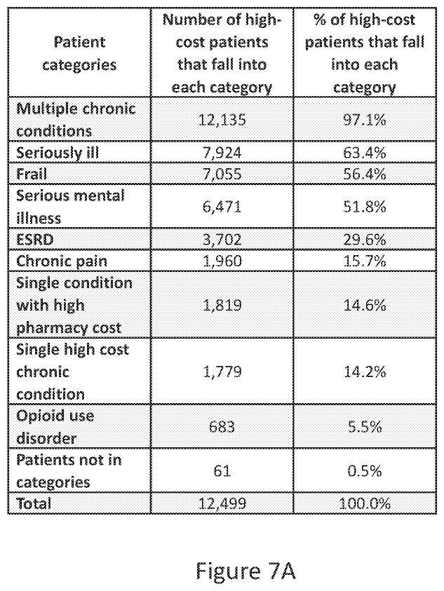

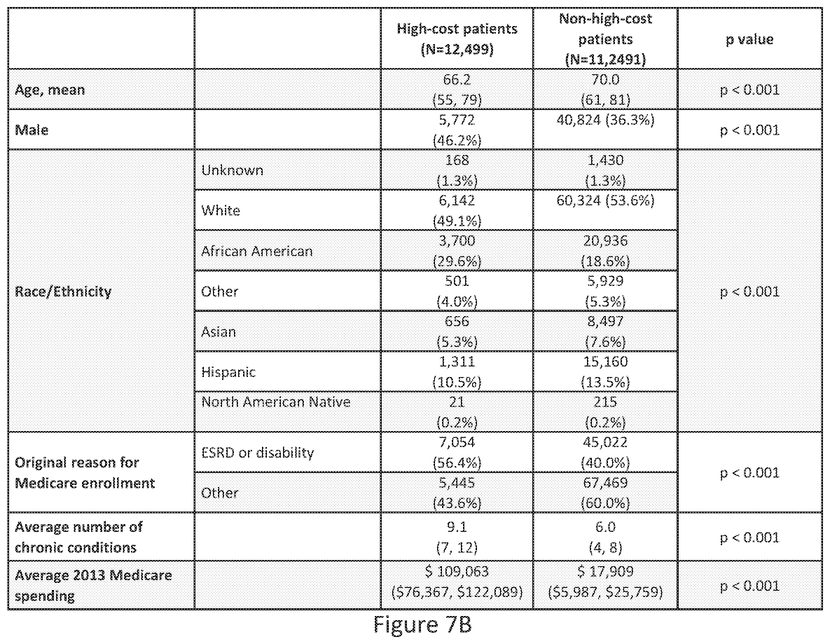

FIG. 7A shows an exemplary distribution of high-cost dual-eligible patients into categories or phenotypes, in accordance with the present embodiments.

FIG. 7B shows exemplary characteristics of the patient population of FIG. 7B, in accordance with the present embodiments. Compared to Medicare fee-for-service (FFS) patients, more high-cost dual-eligible patients are captured by categories.

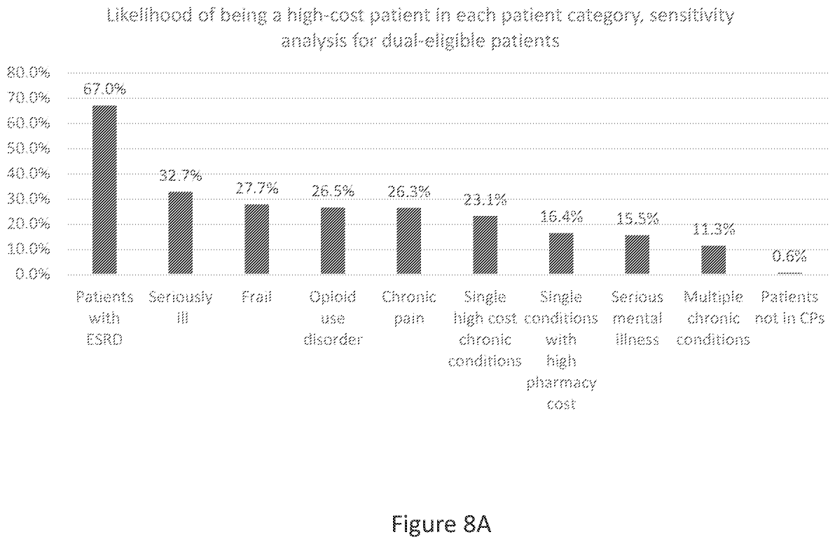

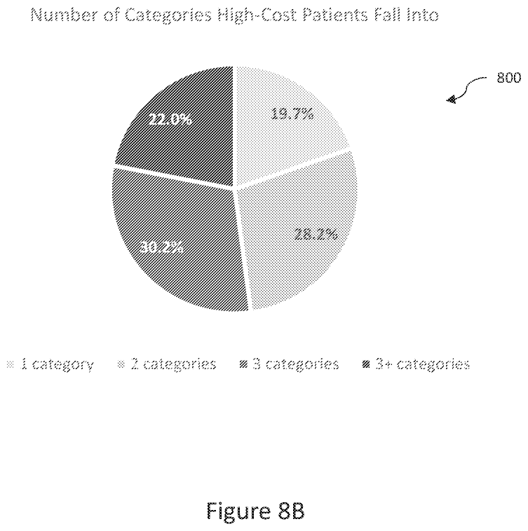

FIG. 8A shows the likelihood of being a high-cost patient in each patient category of an example patient population, in accordance the present embodiments. The likelihood of being a high-cost patient in each patient category is lower in these categories among dual-eligible patients.

FIG. 8B is a chart 800 showing the number of categories each high-cost patient falls into, among the example population of FIG. 8A, in accordance with the present embodiments. As can be seen in the chart, more high-cost dual-eligible patients fall into multiple categories than fall into a single category.

The system developed a novel taxonomy with ten patient categories to identify and categorize high-cost or HNHC Medicare patients. The system found that these patient categories captured over 99% of high-cost or HNHC patients. High-cost or HNHC patients were more likely to have multiple chronic conditions and serious mental illness, or to be seriously ill or frail. In addition, a large proportion of high-cost or HNHC patients also had vulnerable social conditions. The system found the likelihood of patients being high-cost or HNHC in any given category varied significantly: Patients with ESRD were most likely to be high-cost or HNHC patients, followed by those who are seriously ill, frail, or have chronic pain.

The results support a growing understanding of the diversity of high-cost or HNHC patients. High-cost or HNHC patients fall into several, sometimes overlapping categories. Our subgroup analysis also suggests that social risk factors play an important role. Socially vulnerable neighborhoods, such as those with low income and poor housing conditions may be related to high utilization among high-cost or HNHC patients. Taken together, these findings suggest that multiple care models are necessary to meet the unique and varying needs of high-cost or HNHC patients, and that these models should include approaches to address both social and medical complexity.

These findings also suggest that previous definitions and assumptions of high-cost or HNHC patients—which tend to lump them into less nuanced groupings—may not be sufficient to align care models with patient needs. Many studies, for example, have used multiple chronic conditions as a marker for high-cost or HNHC patients, which may not provide sufficient information to target care interventions. The system found that nearly all high-cost or HNHC patients have multiple chronic conditions—as do many patients who are not high-cost or HNHC—so this grouping may not be useful for directing resources in a targeted manner.

The system also found that 70% of high-cost or HNHC patients fall into multiple categories. This suggests that non-mutually exclusive patient categories may be more helpful for designing and implementing care models compared to taxonomies that segment patients into mutually exclusive categories.

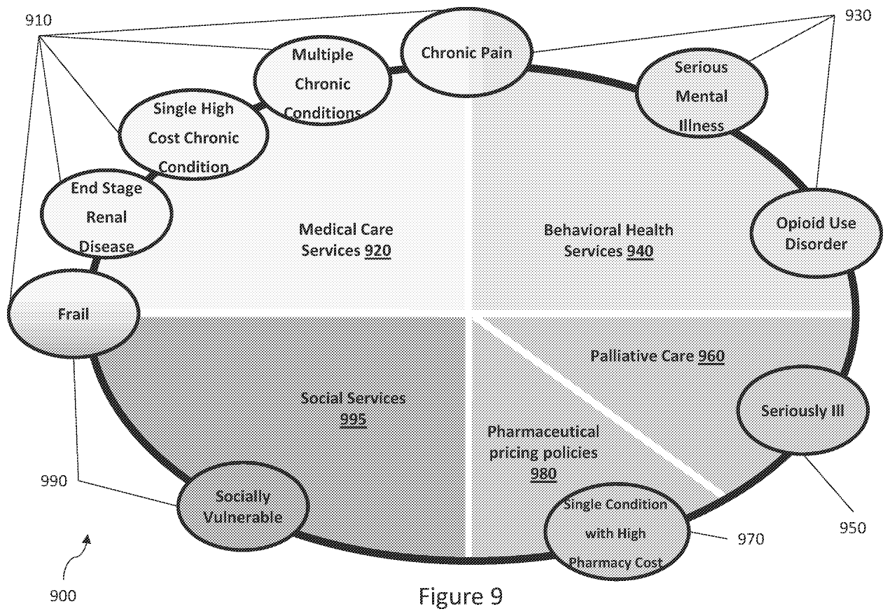

FIG. 9 shows an exemplary mapping of patient categories or phenotypes to action categories, in accordance with the present embodiments. In the example shown in FIG. 9, categories or phenotypes 910 include frail, end stage renal disease, single high-cost chronic condition, multiple chronic conditions, and chronic pain. These categories or phenotypes 910 map to the medical care services action category 920. Similarly, patient categories or phenotypes 930 include chronic pain, serious mental illness, and opioid use disorder, and map to the behavioral health services action category 940. The seriously ill category or phenotype 950 maps to the palliative care action category 960. The “single condition with high pharmacy cost” category 960 maps to the “pharmaceutical pricing policies” action category 980. The frail and socially vulnerable categories or phenotypes 990 map to the social services action category 995.

Categorizing patients into actionable, non-exclusive groups will help to understand their characteristics and align appropriate interventions that fit patients' needs to reduce unnecessary health care spending. For example, patients who are seriously terminally ill could benefit from palliative care. Socially vulnerable patients require services from non-health organizations, such as transportation and housing. Frail patients require both social (e.g. programs to address loneliness) and medical interventions. Patients with opioid use disorder and serious mental illness may need behavioral interventions. Patients with chronic pain may need both behavioral and medical treatments. ESRD, single high cost chronic condition, or multiple chronic conditions groups may need a care manager that could coordinate their intensive medical care service needs. Finally, pharmaceutical pricing policy that control medication prices may be needed for patients having a condition with high pharmacy cost.

The findings further suggest an important role for combining claims, clinical, and social determinants data to develop patient categories. For example, the system found that patients with low albumin levels—a form of clinical data often not captured by claims—had a strikingly higher probability of being high-cost or HNHC patients than other seriously ill and frail patients. Similarly, patients with low BMI or extreme obesity were much more likely to be high-cost or HNHC.

A growing body of evidence also suggests socially disadvantaged individuals are at risk for high healthcare utilization, but the most effective way to measure social vulnerability remains unclear. Researchers seldom have access to detailed individual-level social data, and community level social indices have often been used as a proxy. In this study, the system used SVI to measure the social vulnerability and the system found a large proportion of high-cost or HNHC patients lived in communities with vulnerable social conditions.

The system developed a taxonomy with ten patient categories for high-cost or HNHC Medicare patients. This taxonomy captured most high-cost or HNHC patients and categorized them into clinical meaningful groups. The framework described herein could have important implications for health care delivery and resource allocation by providing a nuanced stratification of high-cost or HNHC patients based on clinical, demographic, and social factors. It may help clinicians and health systems better understand their patient population, identify those at risk for high utilization, and improve care models targeted to their needs.

The identified patient phenotypes 910, 930, 950, 970, and 990 have certain desirable characteristics. First, they collectively capture the vast majority (>99%) of high-cost or HNHC patients. Second, patients within a single phenotype have similar characteristics to one another, and different from those outside the phenotype. Third, membership in the phenotypes is determined quantitatively, by a data-driven analysis, rather than the human judgment of a care provider or care manager. Fourth, the phenotypes have significant predictive value in determining which patients are presently high-cost or HNHC, or will become high-cost or HNHC in the future. Fifth, the phenotypes are non-exclusive, which allows for a much richer and more thorough numerical analysis of patient characteristics and likely outcomes.

In determining patient persistence (e.g., persistently high cost, persistently high utilization, or both), some phenotypes are more important than others (e.g., more likely to result in persistence). Certain combinations of categories (e.g., a patient who is both frail and seriously ill, or who is both seriously ill and has a serious mental illness), predict for very high future utilization. The characteristics of persistent patients are different from those of non-persistent patients, and the identified phenotypes can be effective discriminators between these two categories.

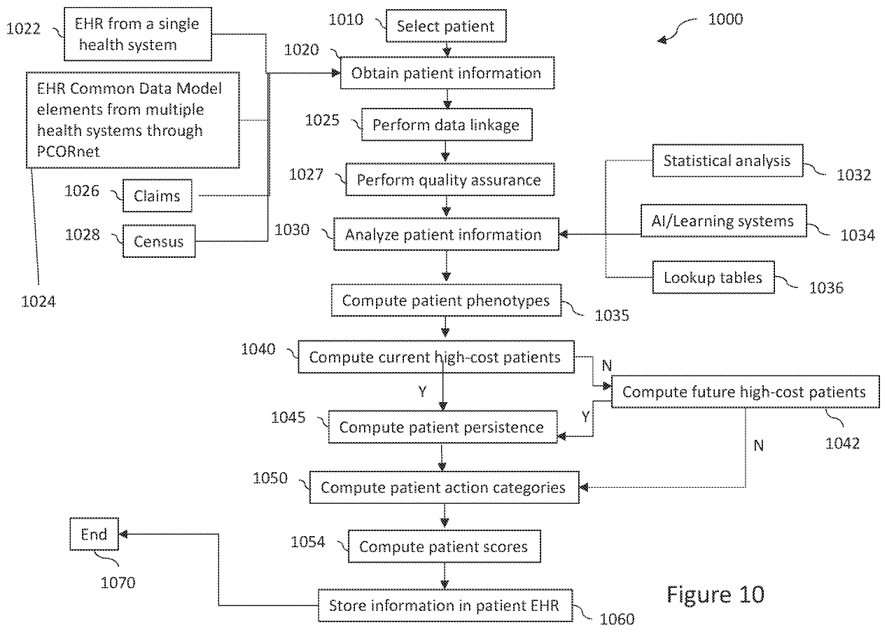

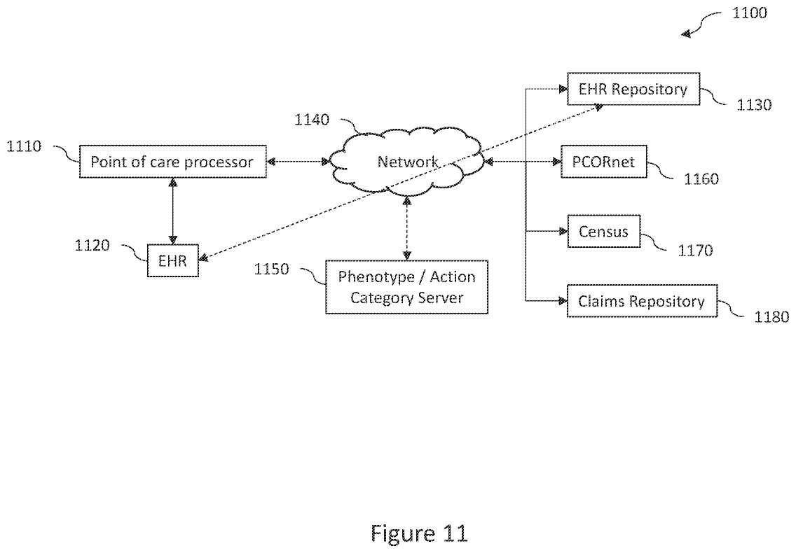

FIG. 10 shows a flow diagram of an example computer-implemented patient classification method 1000, in accordance with the present embodiments. It is understood that the steps of method 1000 may be performed in a different order than shown in FIG. 10, additional steps can be provided before, during, and after the steps, and/or some of the steps described can be replaced or eliminated in other embodiments. One or more of steps of the method 1000 can be carried by one or more devices and/or systems described herein, such as components of the point of care processor 1110 or server 1150 (see FIG. 11), processor circuit 1250, and/or other processor as needed to implement the method.

In step 1010, the method 1000 includes selecting a patient from a patient population.

In step 1020, the method 1000 includes obtaining patient information about the selected patient. Patient information may be drawn from one or more of an electronic health record (EHR) 1022 (which may come from a single health care system such as a care provider's local computing system), or EHR Common Data Model elements from multiple health systems through the National Patient-Centered Clinical Research Network (PCORnet) 1024 (or other equivalent network), claims data (e.g., Medicare, Medicaid, or private insurance claims data) 1026, or census data 1028, or other sources known in the art, or combinations thereof. For example, a patient address or zip code from an EHR 1022 may be used to pull neighborhood data from a census 1028, to derive social vulnerability score as described above.

In step 1025, the method performs data linkage. Developing the patient categories, can require linking of Medicare claims data, EHR data from multiple health systems, and social determinants of health (SDoH) data for over 1 million Medicare patients. The data linkage can be critical as patients may visit various healthcare organizations across geographic regions. In addition, each data source contains unique information (e.g., laboratory test results are only available from EHR data) that represent patient characteristics. Therefore, it is beneficial to combine all patient information to understand a patient's medical, social, and behavioral characteristics that represent their real health needs. Previous work has relied on solely claims or clinical data.

As a single patient may have different identifiers in different healthcare organizations and data sources, the present disclosure includes ensuring accurate data linkage. The linkage of patient EHR data from different health systems may for example be supported at least in part through INSIGHT (the vendor of the EHR data)'s implementation of the Datavant software for de-duplicating and matching patients nationally and locally in a privacy-preserving manner. The Datavant software may not only enhance the accuracy and flexibility of patient matching but may also create opportunities for linking new data sources. An algorithm can for example link EHR data with Medicare claims data. To link SDoH data, the method may geocode patients through a commercial crosswalk to map patients into zip codes, US census block tracts, or other geographic units based on their residential location.

In step 1027, the method performs quality assurance to ensure the algorithm has identified the same patient from both sides (e.g., EHR and Medicare) and linked them together successfully.

In step 1030, the method 1000 includes analyzing the patient information. Analyzing the patient information may include at least one of statistical analysis 1032, including logistic regression, linear regression, or machine learning based methods, such as random forest and gradient boosting 1034, lookup tables 1036, or other analysis methods known in the art, or combinations thereof.

In step 1035, the method computes one or more categories or phenotypes to which the high-cost or HNHC patient belongs. Computing categories or phenotypes for the patient requires comparing all patient information from different data sources, including but not limited to diagnosis, procedures, and demographics, with the definition of each category or phenotype. This usually requires compiling patient data from different data sources and quality assurance to ensure the accuracy of patient information. The method described herein is unique and different from previous work as a patient could fall into multiple categories or phenotypes if his or her conditions are highly complicated. In addition, the present disclosure incorporates patient social determinants of health (SDoH) information when computing categories or phenotypes as SDoH are important drivers of healthcare utilization. Previous studies have focused on medical conditions. It is noted that development of the patient phenotypes (e.g., the ten phenotypes identified herein) requires analysis of data from the identified data sources for a large, statistically significant and statistically representative plurality of patients. For example, such development may require data from hundreds of thousands, millions, tens of millions, or more patients.

In step 1040, the method 1000 determines whether the patient is currently a high-cost or HNHC patient. If yes, execution proceeds to step 1045. If no, execution proceeds to step 1042. This determination may for example require calculating the total healthcare costs a patient has in the entire previous year from all care settings, including but not limited to ambulatory visits, outpatient visits, inpatient visits, post-acute care visits, and long-term care visits. Unlike previous methods, the method disclosed herein may calculate the geographically standardized costs which account for the differences in healthcare prices across geographic regions. Therefore, the calculated healthcare costs more precisely represent patient health needs and utilization, which provides more relevant information to healthcare providers.

In step 1042, the method 1000 determines whether a patient is likely to be a future high-cost or HNHC patient. If yes, execution proceeds to step 1045. If no execution proceeds to step 1050. This determination may for example require predicting the total healthcare costs a patient may have in the upcoming year or upcoming two years, using a predictive statistical model based on other patients from the patient population who have similar characteristics. Alternatively, the determination may simply require predicting the yes or no answer itself, using a predictive statistical model based on similar patients. For example, from a group of past patients with characteristics X and Y, if more than 50% have gone on to become high-cost or HNHC patients, then a current patient may be deemed more than 50% likely to become a high-cost or HNHC patient.

In step 1045, the method 1000 computes patient persistence. For example, the method 1000 may compute whether the patient is persistently high cost (e.g., within the top 10% of patient costs across two or more years), persistently high preventable utilization (e.g., within the top 10% of preventable resource utilization across two or more years), “double persistent” (e.g., both persistently high cost and persistently high preventable utilization), or non-persistent. It is noted that in some populations, double persistent patients represent 26% of all preventable utilization. This information can thus be extremely important to users of the method 1000 to make cost-reducing care decisions about the patient. In some embodiments, this may be a simple arithmetic calculation based on the patient's total costs and utilization, as identified above, from at least two years of past data, although other procedures may be used instead or in addition.

This step may occur for example if the goal of the method is to identify and categorize current high-cost or HNHC patients within a given patient population. In some embodiments, step 1040 does not occur, and execution proceeds directly from step 1030 to step 1045. This may occur for example in cases where the goal of the method is to identify and categorize future high-cost or HNHC patients within a given patient population, regardless of whether they are currently high-cost or HNHC. In other embodiments, step 1040 does occur, but execution then proceeds to step 1045 regardless of the whether or not the patient is currently high-cost or HNHC. This may occur for example if current high-cost or HNHC status is simply another weighted factor to be included in scoring step 1054, as described below.

In step 1050, the method 1000 computes one or more action categories that are appropriate to the categories or phenotypes of the patient. In some embodiments, this computation may be a simple lookup table relating each individual phenotype to an individual action category. However, it is noted that the development of such a lookup table and its contents requires the analysis of patient data as described above, for a large and statistically significant population of patients (e.g., at least hundreds of thousands of patients, and preferably tens of millions or more patients).

In step 1054, the method 1000 computes predictive numerical risk scores for the patient. These may for example include a “future high cost” risk score, a “future high utilization” risk score, a “future high preventable utilization” risk score, a “future high preventable cost” risk score, a “future high cost persistence” risk score, a “future high utilization persistence” risk or a “future double persistence” score. In some cases, two or more of these calculations may be combined to yield an “overall risk score”. Patients with the highest risk scores (e.g., the top 10% or top 20% of risk scores” can then be flagged for the attention of care providers or care managers, as these identified high-risk patients may provide the greatest opportunity for improvements in the overall quality of care and/or reductions in the overall cost of care.

In an example process by which risk scores may be computed, each patient category or phenotype is assigned a weight. Each type of persistence is also assigned a weight, and certain identified combinations are assigned additional weights. Current high-cost status of the patient, as determined in step 1040, may also be assigned a weight. Depending on the outcomes and statistical analysis of a population dataset, different weighting systems may be developed to calculate patient risk. In an example, the weights of each category for the future high-cost patient are:

TABLE 6
Example risk weighting of different phenotypes

| | Weight for Future |
|---|---|
| Categories or Phenotypes | High-Cost Patients |
| End-stage renal disease | 5 |
| Serious mental illness | 1 |
| Opioid use disorder | 1 |
| Single high-cost chronic | 2 |
| condition | |
| Single condition with high | 2 |
| pharmacy costs | |
| Frailty | 2 |
| Seriously ill | 2 |
| Chronic pain | 1 |
| Multiple chronic conditions | 2 |
| Socially vulnerable | 1 |

After the collection of patient information from different sources, and mapping of patients into categories or phenotypes, the risk score for each patient may be calculated for example by summing the weights of each category to which the patient belongs. In an example, the method can then identify patients in the top 10% of the risk score (the highest 10%) as the high risk patients as the priority to target interventions.

These weights may be derived numerically from available data sets of a sufficiently large patient population, such as data sources 1022, 1024, 1026, and 1028 across statistically significant populations. Such data may be referred to as one or more training sets. Weights may for example be derived by traditional statistical analysis, such as logistic regressions, or advanced machine learning methods, such as random forest and gradient boosting. Patient information is analyzed using automated software which in some embodiments may include STATA and R.

In various embodiments, these weights may simply be multiplied by 1 if that phenotype or persistence is present in the patient, and by zero if it is not, with the results then being added up to derive a total risk score. In other embodiments, the phenotypes and persistence serve as inputs to an AI or learning system, and the weights are internal to the AI or learning system may be determined and/or updated dynamically based on training sets. The risk scores may then be the outputs of the AI or learning system.

In various cases, the analysis and branching logic steps described above may take place in real time or near real time, or may occur offline without human intervention, such that the results are visible when a human operator accesses the patient information. In an example, statistical analysis 1032 or AI/learning systems 1034 may determine that the patient is likely to be a high-cost or HNHC patient, and then a combination of statistical analysis 1032 and lookup tables 1036 may determine one or more patient categories or phenotypes, and then a lookup table 1036 may determine one or more action categories that are appropriate to the categories or phenotypes. Statistical analysis 1032 or AI/learning systems 1034 may then determine patient persistence and/or patient scoring. Other analytical combinations are possible, and fall within the scope of the present disclosure.

In step 1060, the method optionally stores the computed phenotypes, action categories, persistence, and/or risk scores in the patient's EHR, or in another data repository where they may be of operational use to care providers or care managers.

In step 1070, the method is complete.

FIG. 11 is a schematic representation, in block diagram form, of an example network architecture 1100 over which the method of FIG. 10 may operate. The network architecture 1100 may include a point of care processor 1110 that may for example be operated by a clinician or clinical assistant. The point of care processor 1110 accesses a patient's EHR, which may be stored locally, or may be stored remotely on an EHR repository 1130 and accessed over a network 1140. The point of care processor 1110 may perform at least some steps of the method 1000, described in FIG. 10. Alternatively or in addition, at least some steps of the method 1000 may be performed by a server 1150 (e.g., a remote, local, distributed, or cloud server), which may stores and/or compute patient phenotypes, action categories, or persistence.

EHR 1120 may be accessed over the network 1140 by either or both of the point of care processor 1110 or the remote server 1150. PCORnet data 1160 or census data 1170 may be accessed over the network 1140 by either or both of the point of care processor 1110 or the remote server 1150. Claims data may be accessible from a claims repository 1180 over the network 1140 by either or both of the point of care processor 1110 or the remote server 1150.

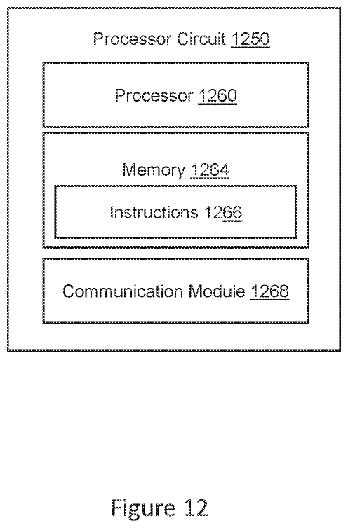

FIG. 12 is a schematic diagram of a processor circuit 1250, according to the present embodiments. The processor circuit 1250 may be implemented in the network architecture 1100, or other devices or workstations (e.g., third-party workstations, network routers, etc.), or on a cloud processor or other remote processing unit, as necessary to implement the method. As shown, the processor circuit 1250 may include a processor 1260, a memory 1264, and a communication module 1268. These elements may be in direct or indirect communication with each other, for example via one or more buses.

The processor 1260 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 1260 may also comprise another hardware device, a firmware device, or any combination thereof configured to perform the operations described herein. The processor 1260 may also be implemented as a combination of computing devices, e.g., a combination of a DSP and a microprocessor, a plurality of microprocessors, one or more microprocessors in conjunction with a DSP core, or any other such configuration.

The memory 1264 may include a cache memory (e.g., a cache memory of the processor 1260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1264 includes a non-transitory computer-readable medium. The memory 1264 may store instructions 1266. The instructions 1266 may include instructions that, when executed by the processor 1260, cause the processor 1260 to perform the operations described herein. Instructions 1266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 1268 can include any electronic circuitry and/or logic circuitry to facilitate direct or indirect communication of data between the processor circuit 1250, and other processors or devices. In that regard, the communication module 1268 can be an input/output (I/O) device. In some instances, the communication module 1268 facilitates direct or indirect communication between various elements of the processor circuit 1250 and/or the network architecture 1100. The communication module 1268 may communicate within the processor circuit 1250 through numerous methods or protocols. Serial communication protocols may include but are not limited to US SPI, I²C, RS-232, RS-485, CAN, Ethernet, ARINC 429, MODBUS, MIL-STD-1553, or any other suitable method or protocol. Parallel protocols include but are not limited to ISA, ATA, SCSI, PCI, IEEE-488, IEEE-1284, and other suitable protocols. Where appropriate, serial and parallel communications may be bridged by a UART, USART, or other appropriate subsystem.

External communication (including but not limited to software updates, firmware updates, preset sharing between the processor and central server, or sensor readings) may be accomplished using any suitable wireless or wired communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

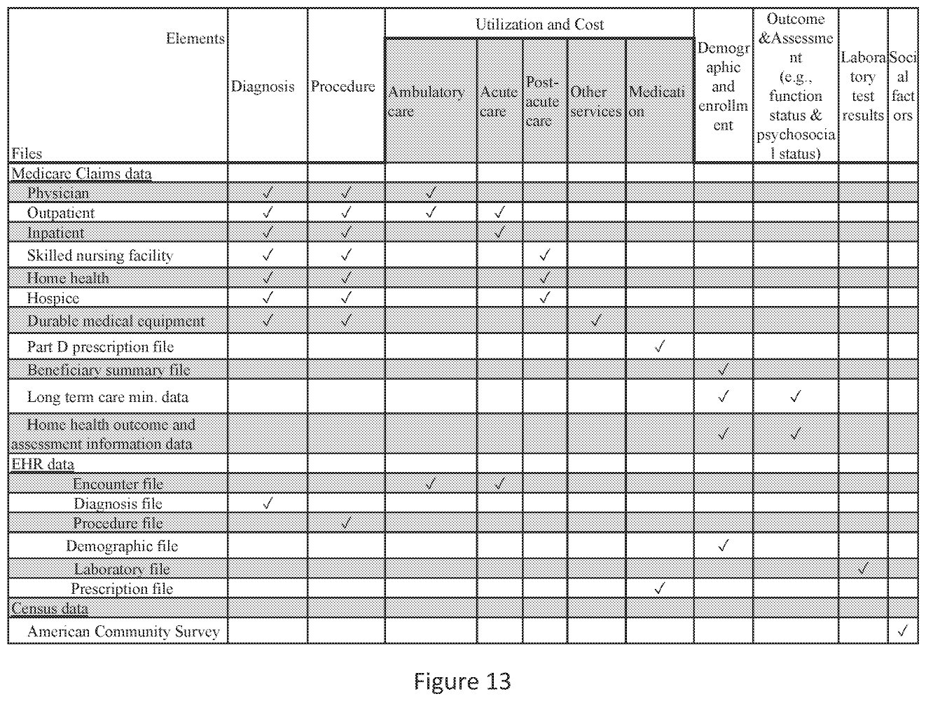

FIG. 13 is a table showing example data types and the example data sources from which they may be available, in accordance with the present embodiments. In an example, analyzing dozens of complex data elements for over 1 million patients requires reducing the volume and complexity of the data by extracting insights and knowledge. This may involve for example searching for particular data types across multiple different data sources, as shown in FIG. 13, searching for multiple different data types across a particular data source, and combinations thereof, and performing statistical analysis on the resulting simplified data set. Through the systems and methods discloses herein, these insights and knowledge can then be applied to individual patients that care providers see on a daily basis, to improve patient outcomes and reduce unnecessary utilization. The reduced data set thus represents a holistic view of patient care across the continuum of care. FIG. 3 illustrates the complexity of the data elements that may be used to develop patient categories or phenotypes as described herein.

As will be readily appreciated by those having ordinary skill in the art after becoming familiar with the teachings herein, the patient classification system described herein advantageously provides systems, methods, and devices for classifying high-cost or high-need high-cost (HNHC) patients into actionable categories that inform and streamline treatment decisions, while also highlighting cost-cutting opportunities available to care providers. The logical operations making up the embodiments of the technology described herein are referred to variously as operations, steps, objects, elements, components, or modules. Furthermore, it should be understood that these may occur or be performed in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the patient classification system. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a ” or “ an ” does not exclude a plurality. Unless otherwise noted in the claims, stated values shall be interpreted as illustrative only and shall not be taken to be limiting.

The above specification, examples and data provide a complete description of the structure and use of exemplary embodiments of the patient classification system as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter. For example, the phenotypes and action categories described above, while providing one illustrative example, are not the only groupings that are contemplated in the present disclosure. Other groupings of the listed conditions/procedures/lab tests, etc. could be selected, and other conditions/procedures/lab tests, etc. could be included, or removed.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

What is claimed is:

1. A computer implemented method of optimizing classification of patient data from large patient datasets, the method comprising:
extracting, from one or more data structures of one or more different data sources, data related to a large plurality of patients;
performing, for each patient of the large plurality of patients, linkage of patient electronic health record (EHR) data across the one or more data sources in a privacy preserving manner to generate patient information for the patient; and
for at least a selected patient of the large plurality of patients:
generating, based on patient information for the selected patient, a high-cost status, a phenotype, and a persistence property of the selected patient, wherein the persistence property is one or both of a persistently high cost or a persistently high utilization; and
applying, to a machine learning model, a high-cost status, a phenotype, and a persistence property to determine at least one risk score for the selected patient, wherein the machine learning model is trained using training data related to the large plurality of patients.

2. The computer-implemented method of claim 1, wherein the at least one risk score comprises at least one of: a future high cost risk score, a future high utilization” risk score, a future high preventable utilization risk score, a future high preventable cost risk score, a future high cost persistence risk score, a future high utilization persistence risk, a future double persistence, or a combination thereof; wherein the computer-implemented method further comprises:
flagging, based on the at least one risk score satisfying a threshold, the selected patient for attention.

3. The computer-implemented method of claim 1, further comprising:
mapping the phenotype to at least one action category for the selected patient; and
storing, into an electronic health record, one or more of the phenotype, the at least one action category, the high-cost status, the persistence property, or the at least one risk score of the selected patient.

4. The computer implemented method of claim 1, wherein extracting comprises:
accessing, via communication over a network, the data structures of the one or more disparate data sources.

5. The computer implemented method of claim 4, wherein the one or more different data sources comprises one or more of: an electronic health record, an insurance claim, National Patient-Centered Clinical Research Network (PCORnet), or census data.

6. The computer implemented method of claim 1, wherein the patient phenotype comprises at least one of socially vulnerable, frail, end stage renal disease, single high-cost chronic condition, multiple chronic conditions, chronic pain, serious mental illness, opioid use disorder, seriously ill, or single condition with high pharmacy cost.

7. The computer implemented method of claim 6, wherein the at least one action category comprises at least one of social services, medical care services, behavioral health services, palliative care, or pharmacological pricing policies.

8. The computer implemented method of claim 6, wherein:
the patient phenotype is socially vulnerable, and the at least one action category comprises social services; or
the patient phenotype if frail, and the at least one action category comprises social services and medical care services; or
the patient phenotype is end stage renal disease, and the at least one action category comprises medical care services; or
the patient phenotype is single high-cost chronic condition, and the at least one action category comprises medical care services; or
the patient phenotype is multiple chronic conditions, and the at least one action category comprises medical care services; or
the patient phenotype is chronic pain, and the at least one action category comprises medical care services and behavioral health services; or
the patient phenotype is serious mental illness, and the at least one action category comprises behavioral health services; or
the patient phenotype is opioid use disorder, and the at least one action category comprises behavioral health services; or
the patient phenotype is seriously ill, and the at least one action category comprises palliative care; or
the patient phenotype is single condition with high pharmacy cost, and the at least one action category comprises pharmaceutical pricing policies.

9. The computer implemented method of claim 1, further comprising:
generating, based on the patient information, a second phenotype of the patient; and
mapping the second phenotype of the patient to a second one or more action categories; and
applying, to a machine learning model, the high-cost status, the second phenotype, and the persistence property to determine an updated risk score for the selected patient.

10. The computer implemented method of claim 1, wherein the high-cost status of the patient comprises “high cost”, “future high cost”, or “non high cost”, and the persistence property of the patient comprises “persistently high cost”, “persistently high preventable utilization”, “persistently high cost and persistently high preventable utilization”, or “non-persistent”.

11. A system for optimizing classification of patient data from large patient datasets, the system comprising:
a processor, and
a memory storing instructions that, when executed by the processor, cause the processor to:
extract, from one or more data structures of one or more different data sources, data related to a large plurality of patients;
perform, for each patient of the large plurality of patients, linkage of patient electronic health record (EHR) data across the one or more different data sources in a privacy preserving manner to generate patient information for the patient; and
for at least a selected patient of the large plurality of patients:
generate, based on patient information for the selected patient, a high-cost status, a phenotype, and a persistence property of the selected patient, wherein the persistence property is one or both of a persistently high cost or a persistently high utilization; and
apply, to a machine learning model, a high-cost status, a phenotype, and a persistence property to determine at least one risk score for the selected patient, wherein the machine learning model is trained using data related to the large plurality of patients.

12. The system of claim 11, wherein the at least one risk score comprises at least one of: a future high cost risk score, a future high utilization” risk score, a future high preventable utilization risk score, a future high preventable cost risk score, a future high cost persistence risk score, a future high utilization persistence risk, a future double persistence, or a combination thereof; wherein the instructions, when executed, further cause the processor to:
flag, based on the at least one risk score satisfying a threshold, the selected patient for attention.

13. The system of claim 11, wherein the instructions, when executed, further cause the processor to:
map the phenotype to at least one action category for the selected patient; and
store, into an electronic health record, one or more of the phenotype, the at least one action category, the high-cost status, the persistence property, or the at least one risk score of the selected patient.

14. The system of claim 11, wherein the instructions, when executed, cause the processor to extract by:
accessing, via communication over a network, the data structures of the one or more disparate data sources.

15. The system of claim 14, wherein the one or more different data sources comprises one or more of: an electronic health record, an insurance claim, National Patient-Centered Clinical Research Network (PCORnet), or census data.

16. The system of claim 11, wherein the patient phenotype comprises at least one of socially vulnerable, frail, end stage renal disease, single high-cost chronic condition, multiple chronic conditions, chronic pain, serious mental illness, opioid use disorder, seriously ill, or single condition with high pharmacy cost.

17. The system of claim 16, wherein the at least one action category comprises at least one of social services, medical care services, behavioral health services, palliative care, or pharmacological pricing policies.

18. The system of claim 16, wherein:
the patient phenotype is socially vulnerable, and the at least one action category comprises social services; or
the patient phenotype if frail, and the at least one action category comprises social services and medical care services; or
the patient phenotype is end stage renal disease, and the at least one action category comprises medical care services; or
the patient phenotype is single high-cost chronic condition, and the at least one action category comprises medical care services; or
the patient phenotype is multiple chronic conditions, and the at least one action category comprises medical care services; or
the patient phenotype is chronic pain, and the at least one action category comprises medical care services and behavioral health services; or
the patient phenotype is serious mental illness, and the at least one action category comprises behavioral health services; or
the patient phenotype is opioid use disorder, and the at least one action category comprises behavioral health services; or
the patient phenotype is seriously ill, and the at least one action category comprises palliative care; or
the patient phenotype is single condition with high pharmacy cost, and the at least one action category comprises pharmaceutical pricing policies.

19. The system of claim 11, wherein the instructions, when executed, further cause the processor to:
generate, based on the patient information, a second phenotype of the patient; and
map the second phenotype of the patient to a second one or more action categories; and
apply, to a machine learning model, the high-cost status, the second phenotype, and the persistence property to determine an updated risk score for the selected patient.

20. The system of claim 11, wherein the high-cost status of the patient comprises “high cost”, “future high cost”, or “non high cost”, and the persistence property of the patient comprises “persistently high cost”, “persistently high preventable utilization”, “persistently high cost and persistently high preventable utilization”, or “non-persistent”.
